(19) United States
(12) Patent Application Publication
(10) Pub. No.: US 2005/0013867 A1
(43) Pub. Date: Jan. 20, 2005

(54) Use of proton sequestering agents in drug formulations

(76) Inventors: S. Russ Lehrman, Los Altos, CA (US); Hi-Shi Chiang, San Jose, CA (US); Mei-Chang Kuo, San Jose, CA (US); Jiang Zhang, San Mateo, CA (US); David Lechuga Ballesteros, Santa Clara, CA (US)

Correspondence Address: Mark A Wilson, 150 Industrial Road, San Carlos, CA, 94070, US

(21) Appl. No.: 10/493,182

(22) PCT Filed: Oct. 16, 2002
(86) PCT No.: PCT/US02/33017

Related U.S. Application Data
(60) Provisional application No. 60/330,074, filed on Oct. 19, 2001.

(51) Int. Cl. A61K 9/14; B29B 9/00

(52) U.S. Cl. 424/489; 264/5

(57) ABSTRACT

Methods are provided for preparing spray-dried, drug-containing particles comprising the steps of: (a) selecting drug, an aqueous solution, and a proton-sequestering agent; (b) adding the drug and the proton-sequestering agent to the solution to form a feed solution; and (c) spray drying the feed solution to form the spray-dried, drug-containing particles, wherein at least a portion of the proton-sequestering agent remains mixed with the drug in the spray-dried, drug containing particles, particles and pharmaceutical formulations comprising the prepared particles as well as methods of use are also provided.

### FIELD OF THE INVENTION

The present invention relates generally to spray-dried, drug-containing particles as well as methods for preparing the particles. More specifically, the particles show improved drug stability profiles. In addition, the invention relates to formulations comprising the particles and methods for treating patients using the spray-dried, drug-containing particles.

### BACKGROUND OF THE INVENTION

Pulmonary delivery of therapeutic proteins is an effective route of administration that offers several advantages over conventional routes of administration. These advantages include, for example, the convenience of patient self-administration, the potential for reduced drug side-effects, the ease of delivery, the elimination of needles, and the like. Many preclinical and clinical studies with inhaled proteins, peptides, DNA and small molecules have demonstrated the efficacy of targeting local, i.e., within the lungs, and systemic delivery of therapeutic proteins.

Despite these initially encouraging results, however, the role of inhalation therapy in the health care field has not grown as expected over recent years, in part due to a set of problems unique to the development of inhalable drug formulations. In particular, dry powder formulations for pulmonary delivery, while offering unique advantages over liquid dosage forms and propellant-driven formulations, are often prone to stability problems. These and other problems considerably diminish the efficiency of delivery and the efficacy of dry powder-based inhalation therapies.

Spray drying is one of several well-known techniques for preparing dry powders. Other techniques include lyophilization, air-drying, spray freeze drying (as described in, for example, U.S. Pat. No. 6,284,282), and co-precipitation spray drying techniques, all of which have been used to prepare micron-sized powders. See, for example, WO 96/32149. Other methods for forming particles based on supercritical fluid technology are also known. See, for example, U.S. Pat. No. 6,063,138. Each technique, however, produces particles that exhibit unsatisfactory properties such as drug instability.

For example, spray drying has been employed with the aim of producing particles suitable for pulmonary inhalation. Spray drying techniques utilize a hot gas stream to evaporate microdispersed droplets created by atomization of a liquid feedstock to form dry powders. While spray drying has been long employed in the food and pharmaceutical industries to prepare dry powders, its application to therapeutic proteins has been rather limited because of the concern that certain proteins may be thermally degraded during the spray drying process. Although there is now a growing body of evidence to support the general utility of spray drying macromolecule-based biotherapeutic formulations to produce biologically active powders suitable for inhalation (as evidenced in WO 98/16205, WO 97/41833, WO 96/32152, WO 96/32116, WO 95/24183, and WO 01/00312), many peptides and proteins, when spray dried, form powders in which the drug is chemically unstable. Due to the loss of drug activity, powders having unstable or unstabilized drugs are unattractive for dry powder inhalation therapy.

Several aspects of the process of particle formation, especially during the spray-drying process, can contribute to drug instability and (in the case of therapeutic proteins) unfolding. Contributors to drug instability include shear stress, high temperatures, exposure to the liquid-air interface (i.e., surface effects), liquid-wall interactions, and the like. Thus, spray drying can result in the formation of dried particles having a relatively high degree of drug inactivity. A therapeutic protein in an acid solution, for example, is particularly prone to degradation during the spray drying process as well as during storage. The present invention is therefore directed to methods for preventing or attenuating degradative processes affecting spray-dried drugs.

### SUMMARY OF THE INVENTION

Accordingly, it is a primary object of the invention to provide a method for preparing spray-dried, drug-containing particles comprising the steps of: (a) selecting a drug, an aqueous solution, and a proton-sequestering agent; (b) adding the drug and the proton-sequestering agent to the solution to form a feed solution, and (c) spray drying the feed solution to form the spray-dried, drug-containing particles, wherein a portion of the proton-sequestering agent remains mixed with the drug in the spray-dried, drug-containing particles.

It is another object of the invention to provide such a method wherein the proton-sequestering agent is selected from the group consisting of amino acids, oligopeptides, short-chain fatty acids, carboxylic acid salts, derivatives thereof, and combinations thereof.

It is an additional object of the invention to provide a spray-dried particle comprising a drug and a proton-sequestering agent, wherein the particle is comprised of a) a core having an outer surface, wherein the core comprises the drug and a first portion of the proton-sequestering agent, and b) an outer layer covering at least a part of the outer surface, wherein the outer layer comprises a second portion of the proton-sequestering agent and is substantially free of the drug.

It is a further object of the invention to provide such a particle wherein the drug is a therapeutic protein.

It is still another object of the invention to provide a formulation comprising a plurality of the spray-dried, drug-containing particles described herein.

It is another object of the invention to provide such a formulation further comprising an excipient.

It is a further object of the invention to provide a method for treating a patient comprising administering, via inhalation, the particles described herein.

Additional objects, advantages and novel features of the invention will be set forth in the description that follows, and in part, will become apparent to those skilled in the art upon the following, or may be learned by practice of the invention.

In one embodiment then, a method for preparing spray-dried, drug-containing particles is provided. The method includes the step of selecting a drug, an aqueous solution, and a proton-sequestering agent. Thereafter, the drug and proton-sequestering agent are added to the aqueous solution to form a feed solution. Once formed, the feed solution is then spray dried to form the spray-dried, drug-containing particles. In the resulting particles, at least a portion and up to 100% of the proton-sequestering agent remains mixed with the drug. When 100% of the proton-sequestering agent remains mixed with the drug, a homogeneous particle results. When less than 100% of the proton-sequestering is mixed with drug, the remaining portion of the proton-sequestering agent forms at least a partial (and up to a complete) outer layer.

With respect to the particle itself, another embodiment of the invention provides a spray-dried particle comprising a drug and a proton-sequestering agent, wherein the particle is comprised of a) a core having an outer surface, wherein the core comprises the drug and a first portion of the proton-sequestering agent, and b) an outer layer covering at least a part of the outer surface, wherein the outer layer comprises a second portion of the proton-sequestering agent and is substantially free of the drug.

Preferably, substantially all of the proton-sequestering agent in the outer layer is in amorphous form, but it can also be in crystalline form. The proton-sequestering agent is believed to sequester drug-degrading protons from the immediate environment of the drug, thereby improving the stability of the drug and extending the storage life for formulations comprising such particles. Dried particles formulated in this way are particularly suitable for pulmonary inhalation.

The ratio of hydrated protons in the feed solution to acidic side chains in the drug is an important factor in determining the rate of protein degradation after spray drying. The addition of a proton-sequestering agent with an ionizable group having a pK that is less than the pH of the solution provides a larger proton “sink” than does an equivalent molar amount of a proton-sequestering agent with a pK that is equal to or higher than the pH of the solution. Similarly, if the pKa of the acidic functional groups bound to the drug is lower than the pK of the proton-sequestering agent, the proton-sequestering agent sequesters more protons than an equivalent molar amount of a proton-sequestering agent with a pK that is lower than pKa of the functional groups. Accordingly, preferred proton-sequestering agents for a given drug are those wherein the pK of the proton-sequestering agent is lower than the pH of the solution and higher than the pI of the drug.

Drug degradation can be measured, for example, by determining the rate of deamidation. Deamidation rates, in turn, provide a measure of the expected stability/storage life of the particles and formulations of the invention. Preferably the proton-sequestering agent improves the storage life of the spray-dried particles by at least 10%, more preferably by at least 25%, and most preferably by at least 50%.

Although any agent that sequesters protons can act as a proton-sequestering agent, it is preferred that the proton-sequestering agent is selected from the group consisting of amino acids, oligopeptides, short-chain fatty acids, carboxylic acid salts, derivatives thereof, and combinations thereof. Although not limited in this regard, any drug prone to degradation can be used in the invention. Preferably, the drug will contain a relatively high percentage of acid-labile groups as such drugs are particularly stabilized with a proton-sequestering agent as described herein. A preferred class of drugs suited for the present methods, particles and formulations are therapeutic proteins.

In another embodiment of the invention, a method for treating a patient is provided based on administering to the patient a formulation comprising the spray-dried, drug-containing particles. The formulations can consist only of the spray-dried, drug-containing particles, or can comprise the spray-dried, drug-containing particles combined with one or more excipients. In this embodiment, a patient suffering from a condition that is responsive to drug therapy is administered, via inhalation, a therapeutically effective amount of the formulation described herein. The spray-dried particles, however, can also be used for other purposes such as being compacted or housed in a unit dosage form for oral or other administration, reconstituted into an aqueous solution and delivered by injection, and so forth.

### DETAILED DESCRIPTION OF THE INVENTION

Before describing the present invention in detail, it is to be understood that this invention is not limited to the particular proton-sequestering agents, spray-drying techniques, drugs, and the like as such may vary. It is also to be understood that the terminology used herein is for describing particular embodiments only, and is not intended to be limiting.

It must be noted that, as used in this specification and the intended claims, the singular forms “a,” “an,” and “the” include plural referents unless the context clearly dictates otherwise. Thus, for example, reference to “a drug” includes a single drug as well as two or more different drugs, reference to a proton-sequestering agent refers to a single proton-sequestering agent as well as two or more different proton-sequestering agents, reference to a “an excipient” refers to a single excipient as well as two or more different excipients, and the like.

In describing and claiming the present invention, the following terminology will be used in accordance with the definitions described below.

The term “amino acid” refers to any molecule containing both an amino group and a carboxylic acid group. Although the amino group most commonly occurs at the position adjacent to the carboxy function, the amino group may be positioned at any location within the molecule. The amino acid may also contain additional functional groups, such as amino, thio, carboxyl, carboxamide, imidazole, and so forth. As used herein, the term “amino acid” specifically includes amino acids as well as derivatives thereof such as, without limitation, norvaline, 2-aminoheptanoic acid, and norleucine. The amino acid may be synthetic or naturally occurring, and may be used in either its racemic or optically active (D-, or L-) forms, including various ratios of stereoisomers. The amino acid can be any combination of such compounds. Most preferred are the naturally occurring amino acids. The naturally occurring amino acids (along with their common abbreviations) are: phenylalanine (phe or F); leucine (leu or L); isoleucine (ile or I); methionine (met or M); valine (val or V); serine (ser or S); proline (pro or P); threonine (thr or T); alanine (ala or A); tyrosine (tyr or Y); histidine (his or H); glutamine (gln or Q); asparagine (asn or N); lysine (lys or K); aspartic acid (asp or D); glutamic acid (glu or E); cysteine (cys or C); tryptophan (trp or W); arginine (arg or R); and glycine (gly or G).

As used herein, “therapeutic protein” is any polymer in which the monomers are amino acids, wherein the polymer has physiological activity upon administration to a patient. Often, but not necessarily, amide bonds link one amino acid monomer to another along the sequence. A “therapeutic protein” may include stereoisomers (e.g., D-amino acids) of the twenty conventional amino acids, unnatural amino acids, and other derivatives known to those skilled in the art. The therapeutic proteins used herein include natural and synthetically or recombinantly derived proteins, as well as analogs thereof, to the extent that they retain at least some degree of physiologic activity.

By “oligopeptide” is meant any polymer in which the monomers are amino acids totaling generally less than about 100 amino acids, preferably less than 25 amino acids. The term oligopeptide also encompasses polymers composed of two amino acids joined by a single amide bond as well as polymers composed of three amino acids.

An “aqueous solvent” refers to water or a mixed solvent system comprising water and one or more water-miscible co-solvents. “Aqueous solution” refers to a solution based on such a solvent. When drug and proton-sequestering agent are combined in the aqueous solution, the resulting solution is referred to as a “feed solution.”

As used herein, a “small-chain fatty acid” includes any molecule having the formulation CH₃(CH₂)ₓCOOH, wherein x is an integer of from about 4 to 30. The term small-chain fatty acid also includes any saturated forms as well as any unsaturated forms, all combination of cis and trans isomers of unsaturated forms, as well as unsubstituted and substituted forms.

“Dry” when referring to a powder (e.g., as in “dry powder”) is defined as containing less than about 10% moisture. Preferred compositions contain less than 7% moisture, more preferably less than 5% moisture, even more preferably less than 3% moisture, and most preferably less than 2% moisture. The moisture of any given composition can be determined by the Karl Fischer titrimetric technique using a Mitsubishi moisture meter Model # CA-06.

An “inhalable” dry powder that is “suitable for pulmonary delivery” refers to a composition comprising solid particles that is capable of (i) being readily dispersed in or by an inhalation device and (ii) inhaled by a subject so that at least a portion of the particles reach the lungs to permit penetration into the alveoli. Such a powder is considered to be “respirable” or “inhalable.”

A “surface active” material is one having surface activity (measured, e.g., by surface tensiometry), as characterized by its ability to reduce the surface tension of the liquid in which it is dissolved.

“Aerosolized” particles are particles which, when dispensed into a gas stream by either a passive or an active inhalation device, remain suspended in the gas for an amount of time sufficient for at least a portion of the particles to be inhaled by the patient, so that a portion of the inhaled particles reaches the lungs. The “emitted dose” or “ED” is a value indicative of a dry powder's degree of aerosolization in a gas stream.

“Fine particle dose” (FPD_{<3.3 μm}) provides a measure of aerosol quality and is defined as the amount of powder which is under 3.3 microns (FPD_{<}3.3 μm) determined by cascade impaction. This parameter corresponds to the total mass under stage 3 of an Andersen impactor when operated at a flow rate of 1 cfm (28.3 L/min) and provides an in vitro estimate of the dose below 3.3 microns delivered to the patient.

“Emitted dose” or “ED” provides an indication of the delivery of a drug formulation from a suitable inhaler device after a firing or dispersion event. More specifically, for dry powder formulations, the ED is a measure of the percentage of powder which is drawn out of a unit dose package and which exits the mouthpiece of an inhaler device. The ED is defined as the ratio of the dose delivered by an inhaler device to the nominal dose (i.e., the mass of powder per unit dose placed into a suitable inhaler device prior to firing). The ED is an experimentally determined parameter, and is typically established using an in vitro device set up to mimic patient dosing. To determine an ED value, a nominal dose of dry powder, typically in unit dose form, is placed into a suitable dry powder inhaler (such as that described in U.S. Pat. No. 5,785,049), which is then actuated, dispersing the powder. The resulting aerosol cloud is then drawn by vacuum from the device, where it is captured on a tared filter attached to the device mouthpiece. The amount of powder that reaches the filter constitutes the emitted dose. For example, for a 5 mg dry powder-containing dosage form placed into an inhalation device, if dispersion of the powder results in the recovery of 4 mg of powder on a tared filter as described above, then the emitted dose for the dry powder composition is: 4 mg (delivered dose)/5 mg (nominal dose)×100=80%. For non-homogenous powders, ED values provide an indication of the delivery of drug from an inhaler device after firing rather than of dry powder, and are based on amount of drug rather than on total powder weight. Similarly for propellant-containing metered-dose inhalers, the ED corresponds to the percentage of drug that is drawn from a dosage form and which exits the mouthpiece of an inhaler device. Emitted dose is used as a measure of dispersibility.

A “dispersible” or “aerosolizable” powder is one having an ED value of at least about 30%, more preferably 40-50%, and even more preferably at least about 50-60% or greater. A powder having superior aerosolizability possesses an ED value of at least about 65% or greater.

“Mass median diameter” or “MMD” is a measure of mean particle size, since the powders of the invention are generally polydisperse (i.e., consisting of a range of particle sizes). MMD values as reported herein are determined by centrifugal sedimentation, although any number of commonly employed techniques can be used for measuring mean particle size (e.g., electron microscopy, light scattering, laser diffraction, and so forth). Instruments suitable for measuring MMD include, for example, the Horiba CAPA-700 particle size analyzer (Horiba Instruments Inc., Irvine, Calif.). “Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, in air, as the particle. The aerodynamic diameter encompasses particle shape, density and physical size of a particle. As used herein, MMAD refers to the midpoint or median of the aerodynamic particle size distribution of an aerosolized powder determined by cascade impaction, unless otherwise indicated. As known to those skilled in the art, an Andersen cascade. Impactor (a sieve-like apparatus with a series of stages that capture particles on plates by inertial impaction according to their size, available from Thermo Anderson, Smyrna, Ga.) or other device can be used to determine MMAD. Preferably, the particles and formulations described herein have an MMAD in the range of between 0.1 μm to 5 μm.

“Pharmacologically acceptable salt” includes, but is not limited to, salts prepared with inorganic acids, such as chloride, sulfate, phosphate, diphosphate, bromide, and nitrate salts, or salts prepared with an organic acid, such as malate, maleate, fumarate, tartrate, succinate, ethylsuccinate, citrate, acetate, lactate, methanesulfonate, benzoate, ascorbate, paratoluenesulfonate, palmoate, salicylate and stearate, as well as estolate, gluceptate and lactobionate salts. Similarly, salts containing pharmacologically acceptable cations include, but are not limited to, lithium, sodium, potassium, barium, calcium, aluminum, and ammonium (including alkyl substituted ammonium).

As used herein, an “excipient” is a nondrug component of a formulation. Generally, the excipient can be included in the aqueous solution, in the feed solution, to the particles, or any combination thereof. Furthermore, in the pulmonary delivery context, an excipient is one that can be taken into the lungs with no significant adverse toxicological effects to the patient.

“Pharmacologically effective amount” or “therapeutically effective amount” is the amount of drug needed to provide a desired therapeutic effect. The exact amount required will vary from subject to subject and will otherwise be influenced by a number of factors, as will be explained in further detail below. An appropriate “effective amount,” however, in any individual case can be determined by one of ordinary skill in the art using only routine experimentation.

As used herein, “pH” is defined as the negative logarithm (base 10) of the hydrogen ion concentration of a solution.

“pI” is the isoelectric point of a molecule, or the pH at which positive and negative charges on the molecule are balanced.

pK” is a measurement of the degree of completeness of a reversible reaction, defined as the negative logarithm (base 10) of the equilibrium constant K; used, for example, to describe the extent of dissociation of a weak acid.

In general, “ambient conditions” are those in which the temperature is between 25° C. and the relative humidity is 60%.

The term “substantially” as in “substantially all of a” component is in a certain form refers to a system in which greater than 50%, more preferably greater than 85%, of the component exists in that form. Similarly, reference to a component that is “substantially amorphous” or “substantially crystalline” refers to a system in which the component is less than 50% in crystalline form and less than 50% in amorphous form, respectively.

The term “patient,” refers to a living organism suffering from or prone to a condition that can be prevented or treated by administration of a drug, and includes both humans an animals.

“Optional” or “optionally” means that the subsequently described circumstance may or may not occur, so that the description includes instances where the circumstance occurs and instances where it does not.

Turning to a first embodiment of the invention, the invention includes a method for preparing spray-dried, drug-containing particles comprising the steps of: (a) selecting a drug, an aqueous solution, and a proton-sequestering agent; (b) adding the drug and the proton-sequestering agent to form a feed solution; and (c) spray drying the feed solution to form the sprary-dried, drug-containing particles, wherein at least a portion of the proton-sequestering agent remains mixed with the drug in the spray-dried, drug-containing particles.

Although solving many problems, the present method for preparing spray-dried, drug-containing particles solves a problem associated with spray-drying drug-containing formulations having a low pH. For example, a spray-dried solution containing parathyroid hormone (6.25% wt. based on total solute) with leucine (93.75% wt. based on total solute) at pH 4 yields particles in which the parathyroid hormone is less stable (i.e., >7% degradation at 40° C. for 13 weeks) than a similar formulation containing 30% wt. (based on total solute) of parathyroid hormone (i.e., <4% degradation at 40° C. for 13 weeks). Degradation of the parathyroid hormone was found as follows: asparagine residues (at positions 10, 16 and 33) and glutamine residues (at positions 6 and 29) released ammonia to form the corresponding aspartic and glutamic acids; methionine sidechains (at positions 8 and 18) oxidized to form the corresponding sulfoxides; and covalent oligomers of parathyroid hormone formed on storage at 40° C. Moreover, it was found that that the glass transition temperatures (T_{g}) of the two formulations were both greater than 100° C., and that the ratio of parathyroid hormone/leucine in the amorphous phase of each formulation was about the same. Thus, neither the mobility of the components nor the relative parathyroid hormone/leucine content accounts for the stability differences observed.

Applicants have now determined, however, that instability for many drugs in the spray-drying context is the result of proton-induced degradation. While not wishing to be bound by theory, it is believed that water and H⁺ (protons) bound to the drug largely account for the observed differences in drug stability.

Asparagine deamidation and methionine oxidation have been characterized in the past. See, for example, Manning et al. (1989) Pharm. Res. 6:903-918. Briefly, asparagine deamidation can occur via two distinct reaction pathways. One pathway, the “nucleophilic attack pathway,” requires two steps: 1) nucleophilic attack of a backbone amide nitrogen on the asparagine sidechain to form a cyclic succinimide, and 2) cleavage of the succinimide ring by water at either of the two carbonyl carbons. The second pathway is direct hydrolysis of the sidechain carboxamide.

The route of sidechain deamidation is highly sensitive to pH. For example, deamidation in aqueous acid occurs via both pathways. It appears, however, that the succinimide formed in the nucleophilic attack pathway is stable, and does not readily hydrolyze to the acid. Although hydrolysis to the acid sidechain does not occur through this route, succinimide ring cleavage apparently does. In aqueous alkali conditions, deamidation only proceeds via the nucleophilic pathway. See Clarke et al. in “Stability of Protein Pharmaceuticals, Part A: Chemical and Physical Pathways of Protein Degradation” the Lability of Asparagine and Aspartic Acid Residues in Proteins and Peptides Ahern et al., Eds.; Plenum: New York, 1992; Chapter 1.

Covalent cross-linking reactions can occur via a mechanism that resembles the nucleophilic attack pathway. Mechanistically, the primary difference between the deamidation and cross-linking reactions is that sidechain functional groups replace water as the nucleophilic agent that opens up the succinimide ring. The pH of the immediate environment will affect the rate of covalent cross-linking. Thus, it is believed that a decrease in water content will increase the rate of cross-linking. See Strickley et al. (1996) Pharm. Res. 13:1142-1153. In addition, covalent cross-linking is likely to be affected by the local concentrations of the nucleophilic sidechains (e.g., the ε-amino group of lysine) and the electrophilic centers (e.g., succinimide).

Consistent with deamidation in acidic media, the parathyroid hormone formulations at pH 4 discussed above undergo deamidation via both pathways. For example, glutamine deamidation (at position 6) is observed, which reflects the nucleophilic attack pathway. Succinimide intermediates, which reflect the pathway associated with cleavage of the succinimide ring, are observed and are likely to be involved in the formation of cross-linked multimers.

Another factor in the degradation process is moisture. Although the spray-drying process removes greater than 99% of the water from the feed solution, some water remains. Thermogravic analysis reveals each gram of a 6.25% (wt. based on total solute) parathyroid hormone/93.75% (wt. based on total solute) leucine particles contains 0.5% (wt.) moisture. Thus, on a molar basis, each gram of the particles contains 2800 mol of water, 7100 mol of parathyroid hormone, and 15 mol of leucine. The water does not randomly distribute, but preferentially binds to the most hydrophilic sites. Table 1, based in part on data provided in Leeder et al. (1974) Colloid Interface Sci. 48(2):339-344, provides the water binding capacities of several functional groups, along with the occurrence of those function groups for a prototypical drug, parathyroid hormone.

TABLE 1
Water Associated With Hydrophilic Groups

| | Relative | Occurrence of |
|---|---|---|
| | Humidity | Functional Groups in |
| Functional group | (5%) | Parathyroid Hormone |
| Carboxyl (asp, glu, C-terminus) | 0.7¹ | 5 |
| Amino (lys, N-terminus) | 0.6 | 4 |
| Guanidino (arg) | 0.6 | 2 |
| Aliphatic alcohol (ser, thr) | 0.05 | 3 |
| Aromatic alcohol (tyr) | 0.16 | 0 |
| Peptide (—CONH—) | 0.04 | 33 |
| Amide (asn, gln) | 0.04 | 5 |
| Heterocyclic imino (pro) | 0.04 | 0 |

¹Moles of water per mole of sorption site.

Table 1 makes clear that amino and carboxyl groups have the highest ability to bind water. Consequently, leucine will be likely highly associated with much of the moisture remaining in the leucine-containing, spray-dried formulations.

Yet another factor in the degradation process is the amorphousness or crystallinity of the system components. For example, in the 6.25% parathyroid hormone formulation discussed above, it is estimated that about 90% of the leucine is crystalline in form. The presence of crystalline leucine decreases the relative amount of amorphous leucine, which, in turn, increases the water concentration in the amorphous phase. As shown in Table 2, the 6.25% parathyroid hormone formulation has a relative abundance of water (2800 μmol) compared to leucine in the amorphous phase (710 μmol) mixed with parathyroid hormone (15 μmol). Therefore, on a molar basis, there is more water than leucine mixed with parathyroid hormone.

The water content in comparison to the water binding capacity can be determined for specific functional groups. Calculations performed using the approximate parameters of Table 1 indicate that there is often excess water in a spray-dried formulation relative to hydrophilic functional groups that are available for binding. Because water will be bound to reactive functional groups in the drug, little or no molecular transport through the solid matrix is necessary for degradation to occur. Applicants believe the association of excess water directly to reactive functional groups in the drug explains the independence of water-induced degradation from glass transition and storage temperatures.

As shown in Table 2, a similar calculation can be made with respect to a 30% (wt. based on total solute) parathyroid and 70% (wt. based on total solute) leucine formulation, which is estimated to have 40% leucine in a crystalline form. In this case, each gram of spray-dried formulation contains 73 μmol of parathyroid hormone and 3200 μmol of leucine. Due to the increase in the amount of amorphous leucine present in the formulation, the calculated water capacity is about five-fold greater. Therefore, the water is expected to be broadly dispersed in the powder, thereby having less of effect on protein degradation.

TABLE 2
Composition of the 6.25% and 30% Parathyroid Hormone (PTH)
Formulations

| | | Estimated | | |
|---|---|---|---|---|
| | 6.25% PTH | water | 30% PTH | Estimated |
| | μmol per gram | binding | μmol per gram | water binding |
| Chemical | of amorphous | capacity | of amorphous | capacity |
| species | powder | (μmol)¹ | powder | (μmol) |
| PTH | 15 | 130 | 73 | 630 |
| Leu | 710 | 920 | 3200 | 4200 |
| H₂O | 2800 (5%)² | — | 1100 (2%)² | — |

¹Calculations based values shown in Table 1.
²Calculations not shown.

As with water, it is possible to calculate the effective concentration and location of protons present in the feed solution. Assuming a 200 ml feed solution without volatilization of acid and containing 1 gram of drug, 20 μmol of hydronium ion-derived protons will be found in the in the spray-dried powder. Like water, the protons associate with the highest affinity sites. For protons, the highest affinity sites are the most basic functional groups, e.g., lysine F-amines, N-terminal α-amines, and histidine sidechains, which will be protonated in the feed solution.

Using parathyroid hormone as an example, the most basic functional groups available for binding protons in this drug are the carboxylate sidechains of aspartic and glutamic acid (pKₐ≈4.8), and the C-terminal carboxylate (pKₐ≈3.5). Henderson-Hasselbach calculations indicate that at pH 4.0, 14% and 76% of the sidechain and C-terminal acid groups are negatively charged, respectively. See Table 3.

TABLE 3
Relative Proton Affinity and Concentration of Basic Functional Groups
in two Parathyroid (PTH) Formulations

| | | | Amount | | Amount |
|---|---|---|---|---|---|
| | | 6.25% | of RCO₂⁻ | 30% | of RCO₂⁻ |
| | | PTH | at pH 4 | PTH | at pH 4 |
| | pKₐ¹ | (μmol) | (μmol) | (μmol) | (μmol) |
| Side chain carboxylate | 4.8 | 60 | 8.4 | 290 | 41 |
| of asp and glu | | | | | |
| C-terminal | 3.5 | 15 | 11 | 73 | 55 |
| carboxylate | | | | | |

¹As with pH, pKₐ reflects the proton affinity as determined in aqueous solution. It is included here as a general indicator of proton affinity.

Combined, these carboxylate functional groups in the 6.25% parathyroid hormone formulation are roughly equivalent to the number of protons available. In the 30% parathyroid hormone formulation, there is about a five-fold excess in the number of carboxylate functional groups. Therefore, in the 30% parathyroid formulation, it is likely that carboxylate functional groups in the protein are not fully neutralized. This results in less protein degradation.

The presence of crystalline leucine in the spray-dried powders appears to significantly increase the amount of water that is in direct contact with parathyroid hormone. In addition, these analyses suggest that the ratio of protons to acidic sidechains in the protein is an important factor in determining the extent of degradation. It is this sequestering of water and protons near the labile functional groups in the drug that is believed to be primarily responsible for the increased degradation rate observed in the 6.25% parathyroid hormone powder as well as in other spray-dried, drug-containing formulations.

The goal of the present invention is to reduce the available concentration of protons in a feed solution. In some cases it will be possible to reduce total proton levels by raising the pH of the solution. Alternatively or in addition, the protons in the feed solution can be sequestered away from reactive sites on the drug by way of adding a proton-sequestering agent. When the drug and the proton-sequestering agent are added to the solution to form a feed solution, the particles formed upon spray drying of the feed solution result in at least a portion of the proton-sequestering agent mixed or integrated with the drug, generally in an amorphous state. The proton-sequestering agent in the dried particle serves to sequester excess protons, thereby stabilizing the protein. A portion of the proton sequestering agents can also form an outer layer at the surface of the droplet, thereby drawing out the protons to protect the drug, which remains in the inner core. In general, the proton sequestering agents of the present invention will have a pKₐ that is lower than the pH of the solution. The difference in pKₐ and pH can be as small as 0.2 and as large as 5 but will preferably be 0.5-1.5 units.

The proton-sequestering agents can be any agent that effectively sequesters protons and the invention is not limited in this regard. Such proton-sequestering agents are known by those of ordinary skill in the art or can be determined through routine experimentation. Generally, however, the proton-sequestering agent for use in the present invention is selected from the group consisting of amino acids, oligopeptides, short-chain fatty acids, carboxylic acid salts, derivatives thereof, and combinations thereof.

Exemplary amino acids and derivatives thereof that can serve as proton-sequestering agents include those selected from the group consisting of glycine, alanine, valine, asparagine, leucine, norleucine, isoleucine, phenylalanine, tryptophan, tyrosine, proline, methionine, acylated forms thereof, and combinations thereof.

Oligopeptides comprising any of the herein described amino acids are also suitable for use as proton-sequestering agents. Preferred oligopeptides, however, include poly-lysine (comprising, for example, 4 to 10 lysine residues), poly-glutamic acid (comprising, for example, 4 to 10 glutamic acid residues), and poly-lysine/alanine (comprising, for example, 2 to 5 residues of lysine and alanine in any sequential order), dileucine, leu-leu-gly, leu-leu-ala, leu-leu-val, leu-leu-leu, leu-leu-ile, leu-leu-met, leu-leu-pro, leu-leu-phe, leu-leu-trp, leu-leu-ser, leu-leu-thr, leu-leu-cys, leu-leu-tyr, leu-leu-asp, leu-leu-glu, leu-leu-lys, leu-leu-arg, leu-leu-his, leu-leu-nor, leu-gly-leu, leu-ala-leu, leu-val-leu, leu-ile-leu, leu-met-leu, leu-pro-leu, leu-phe-leu, leu-trp-leu, leu-ser-leu, leu-thr-leu, leu-cys-leu, leu-try-leu, leu-asp-leu, leu-glu-leu, leu-lys-leu, leu-arg-leu, leu-his-leu, leu-nor-leu, lys-lys-lys, and combinations thereof.

Among short-chain fatty acids that can act as proton-sequestering agents, preferred are those that are liquid at the drying temperature of the aqueous solution. Preferred short-chain fatty acids (alternative names are in provided in parenthesis) include, without limitation: tetradecanoic acid (14:0, myristic acid); pentadecanoic acid (15:0); hexadecanoic acid (16:0, palmitic acid); octadecanoic acid (18:0, stearic acid); eicosanoic acid (20:0, arachidic acid); docsosanoic acid (22:0, behenic acid); tetracosanoic acid (24:0, lignoceric acid); 9-tetradecenoic acid (14:1 n5, myristoleic acid); 9-hexadecenoic acid (16:1 n7, palmitoleic acid); 11-octadecenoic acid (18:1 n7, vaccenic acid); 9-octadenoic acid (18:1 n9, oleic acid); 11-eicosenoic acid (20:1 n9, eicosenoic acid); 5,8,11-eicosatrienoic acid (20:3 n9, mead acid); 13-docosenoic acid (22:1 n9, erucic acid); 15-tetracosanoic acid (24:1 n9, nervonic acid); 9,12,15-octadecatrienoic acid (18:3 n3, alpha-linolenic acid); 6,9,12,15-octadecatetraenoic acid (18:4 n3, stearidonic acid); 11,14,17-eicosatrienoic acid (20:3 n3, ETA); 8,11,14,17-eicsoatetraenoic acid (20:4 n3); 5,8,11,14,17-eicosapentaenoic acid (20:5 n3, EPA); 7,10,13,16,19-docosapentaenoic acid (22:5 n3, DPA); 4,7,10,13,16,19-docosahexaenoic acid (22:6 n3, DHA); 6,9,12,15,18,21-tetracosahexaenoic acid (24:6 n3); 9,12-octadecadienoic acid (18:2 n6, inoleic acid); 6,9,12-octadecatrienoic acid (18:3 n6, gama-linolenic acid); 11,14-eicosadienoic acid (20:2 n6); 8,11,14-eicosatrienoic acid (20:3 n6, homo-gama-linolenic acid); 5,8,11,14-eicosatetraenoic acid (20:4 n6, arachidonic acid); 13,16-docosadienoic acid (22:2 n6); 7,10,13,16-docosatetraenoic acid (22:4 n6); 4,7,10,13,16-docosapentaenoic acid (22:5 n6); 9-trans-hexadecenoic acid (trans 16:1n7, palmitelaidic acid); 9-trans-octadecenoic acid (trans 18:1n9, elaidic acid, ricinoleic acid); 8-eicosaenoic acid (20:1n12); 5-eicosaenoic acid (20:1n15), and combinations thereof.

Carboxylic acid salts can also be used as proton-sequestering agents. Such carboxylic acid salts are small molecules that tend to migrate to the surface of droplets during the spray-drying process drying. Preferred carboxylic acid salts will have a molecular weight 5 to 10 times smaller than the drug. Specific examples of carboxylic acids suitable for use as proton-sequestering agents include, for example, the salts of carboxylic acids selected from the group consisting of acetate, citrate, formate, fumerate, malate, methanoate, proponate, oxalate, benzoate, and succinate.

In addition, notwithstanding any of the specifically mentioned proton-sequestering agents, those proton-sequestering agents that are also “surface active compounds” are preferred. In the present context, such agents are not truly “surfactants,” although surface active compounds do have the property of being polar with a hydrophilic portion and a hydrophobic portion. Without wishing to be bound by theory, it is believed the surface active compound's hydrophobic portion tends to reside at the surface of the droplet while the hydrophilic portion is directed to the inner droplet where water molecules are located. The hydrophilic portion, in turn, attracts protons and effectively “sequesters” them away from the drug. Preferred among the proton-sequestering agents that are surface active compounds are hydrophobic amino acids such as, for example, leucine, isoleucine, glycine, alanine, valine, phenylalanine, proline, methionine, glycine, and combinations thereof.

Due to its surface activity, one particularly preferred amino acid is leucine, which tends to concentrate on the surface of spray-dried particles. That is, the concentration of leucine on the surface of the spray-dried particle is typically greater than elsewhere in the particle. Because leucine has a tendency to crystallize at the surface, it is preferred to use both leucine and a second proton-sequestering agent or other excipient.

Because the proton-sequestering agent tends to reside at the surface of the droplet produced during spray drying, the drug tends to reside in the center of the droplet with the consequence that substantially all of the drug resides in the core upon drying of the droplet. This removes the drug from the air-water interface, which can have a degradative effect on the drug. Moreover, the drug avoids the relatively higher shear forces encountered at the droplet surface during the spray-drying process. With respect to therapeutic proteins, shear forces can unfold the drug leading to a loss of activity. Thus, the addition of the proton-sequestering agent protects and masks the drug from various degradative processes.

It must be remembered that while at least a portion of the proton-sequestering agent without a substantial amount of the drug tends to reside at the surface of the droplet, the proton-sequestering agent does not form a coating, per se. Rather, the portion of the proton-sequestering agent accumulates at a greater concentration on the surface of the droplet during spray drying.

It is also preferred that the molecular weight of the proton-sequestering agent is significantly less than the drug, thereby allowing a greater proportion of drug to be delivered in a given dose. For optimum efficiency, it is preferred that the drug be at least five times the molecular weight of the proton-sequestering agent. In particular, it is preferred that no more than 30% of the weight of a dry particle comprises the proton-sequestering agent when the proton-sequestering agent is an amino acid.

Preferably, the proton-sequestering agent is present in a proportion which is determined by its capacity to sequester protons. This is influenced by a number of factors, including, inter alia, the pK of any acidic groups in the drug, the pK of the proton-sequestering agent, the pH of the aqueous solution, and other factors. Generally, however, the proton-sequestering agent is present in the particle in an amount of from about 1% to about 99% by weight, preferably from about 5% to 98% by weight, and more preferably from about 15 to 95% by weight of the proton-sequestering agent. In addition, the amount of the proton-sequestering agent in any particular circumstance can be determined experimentally, i.e., by preparing compositions containing varying amounts of the proton-sequestering agent, examining the stability and other properties of the solution and spray-dried particles formed from the solution, and determining which formulations have the desired properties, e.g., increased stability.

The drug for use in the present invention is one that can benefit from the addition of a proton-sequestering agent in a solution intended to be spray dried. Typically, such a drug includes functional groups known to be involved with one or more degradative processes. Suitable drugs for use in the present invention include, for example, erythropoietin (EPO), Factor VIII, Factor IX, prothrombin, thrombin, alpha-1 antitrypsin, alglucerase, imiglucerase, cyclosporin, granulocyte colony stimulating factor (GCSF), thrombopoietin (TPO), alpha-1 proteinase inhibitor, elcatonin, calcitonin, granulocyte macrophage colony stimulating factor (GMCSP), human growth hormone (hGH), growth hormone releasing hormone (GHRH), heparin, low molecular weight heparin (LMWH), interferon alpha, interferon beta, interferon gamma, interleukin-1 receptor, interleukin-2, interleukin-1, interleukin-1 receptor antagonist, interleukin-3, interleukin-4, interleukin-6, interleukin-7, interleukin-8, interleukin-9, interleukin-10, interleukin-11, interleukin-12, interleukin-13 receptor, luteinizing hormone releasing hormone (LHRH), leuprolide, nafarelin, goserelin, buserelin, insulin, pro-insulin, insulin analogues (e.g., mono-acylated insulin as described in U.S. Pat. No. 5,922,675), amylin, C-peptide, somatostatin, octreotride, vasopressin, follicle stimulating hormone (FSH), insulin-like growth factor (IGF), insulinotrophin, macrophage-colony stimulating factor (M-CSF), nerve growth factor (NGF), platelet-derived growth factor (PDGF), basic fibroblast growth factor (bFGF), acidic fibroblast growth factor (aFGF), stem cell factor (SCF), oncostatin M, heparin-derived growth factor (HGF), herceptin, epidermal growth factor (EGF), endothelial cell growth factor (ECGF), vascular growth factor (VGF), thyroxin, tissue growth factors, keratinocyte growth factor (KGF), glial growth factor (GGF), tumor necrosis factor (INF), endothelial growth factor, parathyroid hormone (PTH), glucagon, thymosin alpha 1, IIb/IIIa inhibitor, phosphodiesterase (PDE) inhibitors, VLA-4 inhibitors, bisphosphonates, respiratory syncytial virus antibody, cystic fibrosis transmembrane regulator (CFIR) gene, deoxyribonuclease (Dnase), bactericidal/permeability increasing protein (BPI), anti-CMV antibody, any therapeutic monoclonal or polyclonal antibody, pharmacologically acceptable salts of any of the foregoing as well as combinations of any of the foregoing. Particularly suitable for use in the methods and compositions described herein are growth factor hormones, parathyroid hormone, leuprolide, calcitonin, insulin, interferon alpha, interferon beta, interferon gamma, follicle stimulating hormone, luteinizing hormone releasing hormone (LHRH), human growth hormone, pharmacologically acceptable salts thereof, and combinations of any of the foregoing. The therapeutic proteins can be naturally derived or synthesized using recombinant or chemical techniques known to those of ordinary skill in the art. In addition, several therapeutic proteins are available from commercial suppliers such as, for example, Sigma (St. Louis, Mo.).

The amount of the drug in the formulation administered to the patient will typically contain at least about one of the following percentages of active agent: 1%, 5%, 10%, 20%, 30%, 40%, 50%, 60%, 70%, 80%, 90% or more by weight. Preferably, the spray-dried powder will contain at least about 50%, e.g., from about 50 to 99.9% by weight of the drug. For particularly potent drugs, however, low concentrations can be used.

The aqueous solution and or the feed solution can also contain one or more optional excipients, none of which necessarily serves as a charge-increasing excipient. Although the invention is not limited in this regard, such optional excipients preferably include those selected from the group consisting of carbohydrate excipients, inorganic salts, antimicrobial agents, antioxidants, surfactants, buffers, acids, bases, and combinations thereof.

Suitable for use in protecting the drug during spray drying are carbohydrate excipients such as sugars, derivatized sugars such as alditols, aldonic acids, esterified sugars, and sugar polymers. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. Preferred are non-reducing sugars, sugars that can form an amorphous or glassy phase with a drug in a spray-dried solid, and sugars possessing relatively high glass transitions temperatures or “Tgs” (e.g., Tgs greater than 40° C., preferably greater than 50° C., more preferably greater than 60° C., and even more preferably greater than 70° C., and most preferably having Tgs of 80° C. and above). Particularly preferred stabilizing excipients are sucrose, mannitol and trehalose.

The compositions may further include an inorganic salt such as sodium chloride, potassium chloride, sodium sulfate, potassium nitrate, and the like. Salts that provide monovalent or divalent cations such as sodium, potassium, aluminum, manganese, calcium, zinc, and magnesium are preferred. When present, such cations are typically present in relative molar amounts ranging from about 50:1 (cation [mol]/drug [mol] to about 1:1, more preferably between about 20:1 to 2:1).

The formulation may also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the formulation as well. Antioxidants are used to prevent oxidation, thereby preventing the deterioration of the drug. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

The formulation may also include a surfactant in order to facilitate the spray-drying process. Exemplary surfactants include: polysorbates, such as “TWEEN 20” and “TWEEN 80,” and pluronics such as F68 and F88 (both of which are available from BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; and chelating agents, such as EDTA, zinc and other such suitable cations. One preferred excipient combination includes a pluronic (e.g., F68) and trileucine.

Protein excipients, which serve to increase the stability of the drug, may be present in the formulation. Exemplary protein excipients include, without limitation, albumins such as human serum albumin (HSA), recombinant human albumin (rHA), gelatin, casein, hemoglobin, and the like.

Representative buffers, which can be a part of the aqueous solution and/or feed solution, include organic acid salts of citric acid, ascorbic acid, gluconic acid, carbonic acid, tartaric acid, succinic acid, acetic acid, or phthalic acid. Other suitable buffers include Tris, tromethamine hydrochloride, borate, glycerol phosphate and phosphate.

Acids or bases may be added to the feed and solution in order to adjust the pH according to the desires of the formulator. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

Preferably, although not necessarily, permeation enhancers (e.g., dimethylsulfoxide) and buffers are not present in the solution or final formulation administered to the patient.

Other optional excipients suitable for use in the compositions according to the invention are listed in “Remington: The Science & Practice of Pharmacy,” 19^{th} ed., Williams & Williams, (1995), “Physician's Desk Reference, 52^{nd} ed., Medical Economics, Montvale, N.J. (1998), WO 96/32096, and in “Handbook of Pharmaceutical Excipients,” 3^{rd} ed., Kibbe, A. H. Editor (2000).

The amount of any individual excipient (when present) in the solution or in the final formulation administered to the patient will vary depending on the activity of the excipient and particular needs of the formulation. Typically, the optimal amount of any individual excipient is determined through routine experimentation, i.e., by preparing compositions containing varying amounts of the excipient (ranging from low to high), examining the stability, MMADs and dispersibilities of the resulting spray-dried compositions, and then further exploring the range at which optimal aerosol performance is attained with no significant adverse effects.

Generally, however, the excipient will be present in the solution or formulation administered to the patient in an amount of about 1% to about 99% by weight, preferably from about 5%-98% by weight, more preferably from about 15-95% by weight of the excipient, with concentrations less than 30% by weight most preferred.

Once the drug, the aqueous solution, the proton-sequestering agent and any optional excipient(s) have been selected, the drug, the proton-sequestering agent and the optional excipients (when present) are added to the solution to form a feed solution for spray drying. In addition, the excipient(s) can be added to the feed solution. In either case, the feed solution is typically mixed well prior to spray drying. Preferably, the drug is dissolved in the aqueous solution. The pH range of the drug-containing solution is generally between about 3 and 7, more typically between about 3 to 5, and most preferably between about 3.5 to 4.

The solution can optionally contain water-miscible solvents, such as acetone, alcohols and the like. Representative alcohols suitable for this purpose include lower alcohols such as methanol, ethanol, propanol, and isopropanol. Such mixed solvent systems typically contain from about 0-80% of the water miscible solvent, more preferably from about 20-40%, and most preferably from about 10-30% of the water miscible solvent. The feed solution will generally contain solids dissolved at a concentration from 0.01% (weight/volume) to about 20% (weight/volume), usually from 0.05% to 10% (weight/volume), and preferably from about 0.1 to 2% (weight/volume). In particular, the feed solution will typically possess one of the following solids concentrations: 0.1 mg/ml or greater, 0.5 mg/ml or greater, 1 mg/ml or greater, 1.5 mg/ml or greater, 2 mg/ml or greater, 3 mg/ml or greater, 4 mg/ml or greater, or 5 mg/ml or greater. When the drug is a protein, the protein can be spray dried at a solids concentration of 0.1 mg/ml, which is effective to provide a spray-dried solid in which conformation of the native protein is preserved. Preferably, however, the maximum amount of solids content will be used when the drug is a therapeutic protein so that relatively high amounts of the protein are found in each droplet, thereby decreasing the potential for denaturing. It is believed that the likelihood of denaturing increases when the protein molecules have access to the air-liquid interface.

Once the components have been combined, the feed solution is spray dried according to conventional spray-drying techniques. Spray drying of the feed solution can be carried out, for example, as described in “Spray Drying Handbook,” 5^{th} ed., K. Masters, John Wiley & Sons, Inc., NY, N.Y. (1991), WO 97/41833 and WO 96/32149.

For example, the solutions can be spray dried in a conventional spray drier, such as those available from commercial suppliers such as Niro A/S (Denmark), Buchi (Switzerland) and the like, resulting in a dispersible, dry powder. Optimal conditions for spray drying the solutions will vary depending upon the solution components, and are generally determined experimentally. The gas used to spray dry the material is typically air, although inert gases such as nitrogen or argon are also suitable. Moreover, the temperature of both the inlet and outlet of the gas used to dry the sprayed material is such that it does not cause decomposition or degradation of the drug in the sprayed material. Such temperatures are typically determined experimentally, although generally, the inlet temperature will range from about 50° C. to about 200° C., while the outlet temperature will range from about 30° C. to about 150° C. Preferred parameters include atomization pressures ranging from about 20 to 150 psi (0.14 to 1.03 MPa), and preferably from about 30-40 to 100 psi (0.21-0.28 to 0.69 MPa). Typically the atomization pressure employed will be one of the following: 20 psi (0.14 MPa), 30 psi (0.21 MPa), 40 psi (0.28 MPa), 50 psi (0.34 MPa), 60 psi (0.41 MPa), 70 psi (0.48 MPa), 80 psi (0.55 MPa), 90 psi (0.62 MPa), 100 psi (0.69 MPa), 110 psi (0.76 MPa), 120 psi (0.83 MPa) or above.

The spray-dried, drug-containing particles formed by the method represent an additional embodiment of the invention. Each particle comprises a drug and a proton-sequestering agent, wherein the particle is comprised of a) a core having an outer surface, wherein the core comprises the drug and a first portion of the proton-sequestering agent, and b) an outer layer covering at least a part of the outer surface, wherein the outer layer comprises a second portion of proton-sequestering agent and is substantially free of the drug.

Within each particle, the drug is present in an amount of at least about 1% by weight and the total amount of the proton-sequestering agent is present in an amount not greater than 30% by weight. Preferably, however, the drug is present in an amount of at least about 50% by weight and the total amount of the proton-sequestering agent is present in an amount not greater than 30% by weight.

The proton sequestering agent, drug, and optional excipient can exist in either crystalline or amorphous forms. It is preferred that substantially all of the drug is present in each particle in an amorphous form. Preferably, substantially all of the portion of the proton-sequestering agent in the core is in an amorphous form. As for the portion of the proton-sequestering agent in the outer layer, substantially all of this portion can be in either a crystalline or amorphous form. In addition, the particle may also include a transition zone disposed between the core and outer layer. The transition zone is preferably comprised of an amorphous form of the proton-sequestering agent, a crystalline form of the proton-sequestering agent, and an amorphous form of the drug.

The invention also provides for pharmaceutical formulations comprising the spray-dried, drug-containing particles described herein. Although preferably suited for inhalation therapy, the formulations can be used for other purposes as well, e.g., formulation for inclusion in a gelatin capsule for oral therapy. With the exception of buffers, acids and bases, one or more of the above-described excipients are optionally present in the pharmaceutical formulation. In this case however, the excipient is added to the particles rather than to the aqueous solution and/or feed solution. The same excipient added to the aqueous solution and/or feed solution or a completely different excipient can be added to the particles. Preferred excipients that may be added to particles include, for example, carbohydrate excipients, inorganic salts, antimicrobial agents, antioxidants, surfactants, and combinations thereof.

The drug-containing, spray-dried particles generally have a mass median diameter (MMD) of less than about 20 μm, preferably less than about 10 μM, more preferably less than about 7.5 μm, and still more preferably less than about 4 μm, with mass median diameters less than about 3.5 μm being most preferred. Expressed in a range, the drug-containing, spray-dried particles are preferably in the range of about 0.1 μm to 5 μm in diameter, preferably from about 0.2 to 4.0 μm. When an optional excipient is added to the drug-containing, spray-dried particles, the excipient can have the same size as the spray-dried particles, although the particle size of any excipient can also be larger and nonrespirable. With respect to the later, a carbohydrate carrier such as lactose serving as a carrier may have a particle size of about greater than 40 microns in size can be added to drug-containing, spray-dried particles produced in accordance with the invention.

The particles and pharmaceutical formulations of the invention may further be characterized by density. The particles and powder formulations will generally possess a bulk density of from about 0.1 to 10 g/cm³, preferably from about 0.1 to 2 g/cm³, and more preferably from about 0.15 to 1.5 g/cm³.

The particles and pharmaceutical formulations will generally have a moisture content below about 20% by weight, usually below about 10% by weight, and preferably below about 6% by weight. More preferably, the particles and powder formulations will typically possess a residual moisture content below about 3%, more preferably below about 2%, and most preferably between about 0.5 and 2% by weight. Such low moisture-containing solids tend to exhibit a greater stability upon packaging and storage. Generally, the particles of powder formulations of the invention are hygroscopic, i.e., moisture absorbing. Therefore, the particles and powder formulations can be stored in sealed containers such as blister packages to prevent hygroscopic growth.

An additional measure for characterizing the overall aerosol performance of particles and pharmaceutical formulations is the fine particle fraction (FPF), which describes the percentage of powder having an aerodynamic diameter less than 3.3 microns. The particles and pharmaceutical formulations are particularly well suited for pulmonary delivery, and possess PPF values ranging from about 30% to 64% or more. Preferred particles and pharmaceutical formulations contain at least about 30 percent of aerosol particle sizes below 3.3 μm to about 0.5 μM and are thus extremely effective when delivered in aerosolized form.

The particles and pharmaceutical formulations described herein also possess chemical and physical stability over time. Generally, with respect to chemical stability, the drug contained in the formulation will degrade by no more than about 10% upon spray drying. Stated differently, the drug-containing, spray-dried particles possess at least about 90% intact drug, preferably at least about 95% intact drug, and even more preferably will contain at least about 97% intact drug.

Moreover, the particles and pharmaceutical formulations of the invention further demonstrate good stability upon storage. For example, when the drug is a therapeutic protein, the total protein aggregate content is less than 10% after storage for one month at 40° C. and ambient relative humidity. With respect to aerosol performance, the particles and pharmaceutical formulations exhibit a drop in emitted dose of no more than about 20%, preferably no more than about 15%, and more preferably by no more than about 10%, when stored under ambient conditions for a period of three months.

The improvement in aerosol properties noted for the particles and pharmaceutical formulations results in several related advantages, such as: (i) reducing costly drug losses to the inhalation device, since more powder is aerosolized and is therefore available for inhalation by a patient; (ii) reducing the amount administered, due to high aerosolization efficiency, and (iii) reducing the number of inhalations per day by increasing the amount of aerosolized drug that reaches the lungs of a patient.

The invention also provides a method for treating a patient suffering from a condition that is responsive to treatment with the drug. The method of treatment involves administering to the patient, via inhalation, formulations comprising the described particles (either alone or combined with one or more excipients added after the formation of the spray-dried particles). The method of treatment may be used to treat any condition that can be remedied or prevented by administration of the particular drug. Those of ordinary skill appreciate which conditions a specific drug can effectively treat. The actual dose to be administered will depend upon the age, weight, and general condition of the subject as well as the severity of the condition being treated, the judgment of the health care professional, and specific drug being used. Therapeutically effective amounts are known to those skilled in the art and/or are described in the pertinent reference texts and literature. Generally, an effective amount will range from about 0.001 mg/day to 100 mg/day, preferably in doses from 0.01 mg/day to 75 mg/day, and more preferably in doses from 0.10 mg/day to 50 mg/day.

The particles and pharmaceutical formulations described herein may be administered using any suitable dry powder inhaler (DPI). Briefly, some DPIs utilize the patient's inhaled breath as a vehicle to transport the dry powder drug to the lungs. Generally, the powder is contained in a receptacle having a puncturable lid or other access surface, preferably a paper or foil surface of a blister package or cartridge, where the receptacle may contain a single dosage unit or multiple dosage units. Each dose may be weighed separately using a conventional scale. In addition, convenient methods are available for filling large numbers of cavities (i.e., unit dose packages) with metered doses of dry powder medicament. See, for example, WO 97/41031. For a description of various DPIs and how they work, reference is made to U.S. Pat. Nos. 5,458,135, 5,740,794, and 5,785,049, and WO 01/00263.

Other types of DPIs suitable for delivering the particles and pharmaceutical formulations described herein include those that use a hard gelatin capsule containing a premeasured dose. See, for example, U.S. Pat. Nos. 3,906,950 and 4,013,075.

Other dry powder dispersion devices for pulmonary administration include those described in, for example, European Patent Nos. EP 129985, EP 472598, and EP 467172 and U.S. Pat. No. 5,522,385. Also suitable is the TURBUHALER device available from Astra-Draco. This type of device is described in detail in U.S. Pat. Nos. 4,668,281, 4,667,668, and 4,805,811. Other suitable devices include dry powder inhalers such as the Rotahaler® (Glaxo), Discus® (Glaxo), Spiros™ Aura Pharmaceuticals), and Spinhaler® (Fisons) inhalers. Also suitable are devices that use a piston to provide air for either entraining powdered medicament, lifting the medicament from a carrier screen by passing air through the screen, or mixing air with powder medicament in a mixing chamber with subsequent introduction of the medicament to the patient through the mouthpiece of the device. See, for example, U.S. Pat. No. 5,388,572.

The particles or pharmaceutical formulations may also be delivered using a pressurized, metered dose inhaler (MDI). The particles or powder formulation are dissolved or suspended in a pharmaceutically inert liquid propellant, e.g., a chlorofluorocarbon, fluorocarbon or hydrogen-containing fluorocarbon. See, for example, U.S. Pat. Nos. 5,320,094 and 5,672,581. In addition, the particles and pharmaceutical formulations described herein can be dissolved or suspended in a solvent, e.g., water, ethanol or saline, and administered by nebulization. Nebulizers for delivering an aerosolized solution include the AERx™ (Aradigm), the Ultravent® (Mallinkrodt), and the Acorn II® (Marquest Medical Products) devices.

Prior to use, the particles and pharmaceutical formulations are generally stored under ambient conditions, and preferably are stored at temperatures at or below about 25° C., and relative humidities (RH) ranging from about 30 to 60%. More preferred relative humidity conditions, e.g., less than about 30%, can be achieved by incorporating a desiccating agent in the secondary packaging of the dosage form. Particles and powder formulations may also be stored under “accelerated” stability at 40° C., relative humidity 75%, for the purpose of determining stability.

The following examples are illustrative of the present invention, and are not to be construed as limiting the scope of the invention. Variations and equivalents of this example will be apparent to those of skill in the art in light of the present disclosure, the drawings and the claims herein.

All articles, books, patents and other publications referenced herein are hereby incorporated by reference in their entirety.

### EXPERIMENTAL

The practice of the invention will employ, unless otherwise indicated, conventional techniques of pharmaceutical formulating and the like, which are within the skill of the art. Such techniques are fully explained in the literature. See, for example, Remington, The Science and Practice of Pharmacy, supra.

In the following examples, efforts have been made to ensure accuracy with respect to numbers used (e.g., amounts, temperatures, etc.) but some experimental error and deviation should be accounted for. Unless indicated otherwise, temperature is in degrees C. and pressure is at or near atmospheric pressure at sea level. All reagents were obtained commercially unless otherwise indicated.

### Example 1

The following deamidation study was performed for several formulations having different excipients, at various pH levels, temperatures, and storage times. The results are provided in Table 4. Lower values in the table, e.g., 0.21, represent low levels of deamidation, whereas the higher levels, e.g., 2.5, represent a high levels of deamidation. Although each formulation may be prone to other sources of degradation, the data show the advantages of the present invention. The solutions were stored in the solid state.

TABLE 4
Percent Total Deamidation for Various Parathyroid Hormone (PTH) Formulations

| | % Total Deamidation | | | | | | | | | |
|---|---|---|---|---|---|---|---|---|---|---|
| | | t = 1 wk | | | t = 2 wks/3 wks | | | t = 4 wks | | |
| Formulation | t = 0 | 5° C. | 25° C. | 40° C. | 5° C. | 25° C. | 40° C. | 5° C. | 25° C. | 40° C. |
| 30% PTH, 70% | 0.23 | 0.23 | 0.26 | 0.31 | 0.21 | 0.31 | 0.36 | 0.33 | 0.37 | 0.61 |
| leucine (pH) 4.0 | | | | | | | | | | |
| 6.25% PTH, | 0.24 | 0.21 | 0.29 | 1.06 | 0.23 | 0.62 | 1.11 | 0.33 | 0.82 | 2.58 |
| 93.75% leucine | | | | | | | | | | |
| (pH 4.0) | | | | | | | | | | |
| 6.25% PTH, 10% | 0.20 | 0.23 | 0.27 | 0.54 | 0.22 | 0.30 | 1.35 | 0.33 | 0.53 | 1.92 |
| sucrose, 88.75% | | | | | | | | | | |
| leucine (pH 4.0) | | | | | | | | | | |
| 6.25% PTH, 25% | 0.19 | 0.22 | 0.28 | 0.31 | 0.26 | 0.25 | 0.35 | 0.35 | 0.38 | 0.55 |
| trileucine, 68.75% | | | | | | | | | | |
| leucine (pH 4.0) | | | | | | | | | | |
| 6.25% PTH, | 0.22 | 0.23 | 0.26 | 0.27 | 0.22 | 0.26 | 0.22 | 0.27 | 0.33 | 0.28 |
| 93.75% sucrose | | | | | | | | | | |
| (pH 4.0) | | | | | | | | | | |
| 6.25% PTH, 10% | 0.37 | 0.40 | 0.31 | 0.97 | 0.42 | 0.59 | 1.28 | 0.48 | 0.71 | 1.69 |
| citrate, 83.75% | | | | | | | | | | |
| leucine (pH 4.0) | | | | | | | | | | |
| 6.25% PTH, 10% | 0.50 | 0.97 | 0.49 | 0.34 | | | | | | |
| citrate, 83.75% | | | | | | | | | | |
| leucine (pH 5.2) | | | | | | | | | | |
| 6.25% PTH, 10% | 0.22 | 0.25 | 0.23 | 0.24 | 0.85 | 0.33 | 0.30 | 0.33 | 0.31 | 0.38 |
| citrate. 83.75% | | | | | | | | | | |
| leucine (pH 6.5) | | | | | | | | | | |
| 6.25% PTH, 10% | 0.49 | 0.86 | 0.51 | 0.28 | | | | | | |
| citrate, 3% | | | | | | | | | | |
| mannitol, 80.75% | | | | | | | | | | |
| leucine (pH 5.2) | | | | | | | | | | |
| 6.25% PTH, | 0.26 | 0.26 | 0.30 | 0.32 | 0.24 | 0.22 | 0.34 | 0.35 | 0.33 | 0.40 |
| 93.75% mannitol | | | | | | | | | | |
| (pH 4.7) | | | | | | | | | | |
| 6.25% PTH, 28.7% | 0.24 | 0.24 | 0.22 | 0.25 | 0.20 | 0.23 | 0.43 | 0.33 | 0.32 | 0.32 |
| citrate, 65.05% | | | | | | | | | | |
| leucine (pH 6.5) | | | | | | | | | | |
| 6.25% PTH, 25% | 0.22 | 0.23 | 0.25 | 0.81 | 0.22 | 0.24 | 0.64 | 0.30 | 0.35 | 0.59 |
| trileucine, 0.5% | | | | | | | | | | |
| methionine, | | | | | | | | | | |
| 68.75% leucine | | | | | | | | | | |
| (pH 4.0) | | | | | | | | | | |

### Examples 2-6

To control the degradation rate of parathyroid hormone by decreasing the amount of protons (and water) relative to the amount of the drug, the following formulations can be used. Each formulation can be spray dried using standard conditions.

### Example 2

An 80% (wt.) parathyroid hormone/20% (wt.) leucine formulation is prepared at pH 4, having a 0.5% (wt.) total solids with a volume of 50 mL. The resulting powder will contain 200 mg (49 μmol) parathyroid hormone, 50 mg (12 μmol) leucine, and 5 μmol of acid.

### Example 3

An 8% (wt.) parathyroid hormone/72% (wt.) sucrose/20% (wt.) leucine formulation is prepared at pH 4, having a 0.5% (wt.) total solids with a volume of 50 mL. The resulting powder will contain 20 mg (4.9 μmol) parathyroid hormone, 180 mg (526 μmol) sucrose, 50 mg (12 μmol) leucine, and 5 μmol of acid.

### Example 4

A 0.8% (wt.) parathyroid hormone/79.2% (wt.) sucrose/20% (wt.) leucine formulation is prepared at pH 4, having a 0.5% (wt.) total solids with a volume of 50 mL. The resulting powder will contain 2 mg (0.49 μmol) parathyroid hormone, 198 mg (579 μmol) sucrose, 50 mg (12 μmol) leucine, and 5 μmol of acid.

### Example 5

A 0.8% (wt.) parathyroid hormone/79.2% (wt.) sucrose/20% (wt.) leucine formulation is prepared at pH 6, having a 0.5% (wt.) total solids with a volume of 50 mL. The resulting powder will contain 2 mg (0.49 μmol) parathyroid hormone, 198 mg (579 μmol) sucrose, 50 mg (12 μmol) leucine, and 0.05 μmol of acid.

### Example 6

A 0.8% (wt.) parathyroid hormone/77.2% (wt.) sucrose/20% (wt.) leucine/2% (wt.) disodium citrate formulation is prepared at pH 4, having a 0.5% (wt.) total solids with a volume of 50 mL. The resulting powder will contain 2 mg (0.49 μmol) parathyroid hormone, 193 mg (564 μmol) sucrose, 50 mg (12 μmol) leucine, 5 mg (21 μmol) disodium citrate, and 5 μmol of acid.

### Example 7

To demonstrate that the proton-sequestering agents decrease the degradation rate of a drug such as parathyroid hormone, the following procedure is carried out.

Determine the degradation rates of parathyroid hormone (1 mg/mL; 0.24 mM)±disodium citrate at pH 3 at 40° C. In separate aliquots, incubate with the following amounts of disodium citrate (5.73 μg/mL, 24 mM; 57.3 μg/mL, 0.24 mM; and 5.73 μg/mL, 0.024 mM). Include a control aliquot containing parathyroid hormone lacking disodium citrate. High pressure liquid chromatography is used to measure the degradation of parathyroid hormone at various time points. The results demonstrate that parathyroid hormone retains more activity in the citrate solution.

1. A method for preparing spray-dried, drug-containing particles comprising the steps of:
(a) selecting a drug, an aqueous solution, and a proton-sequestering agent;
(b) adding the drug and the proton-sequestering agent to the solution to form a feed solution; and
(c) spray drying the feed solution to form the spray-dried, drug-containing particles, wherein at least a portion of the proton-sequestering agent remains mixed with the drug in the spray-dried, drug-containing particles.

2. The method of claim 1, wherein the molar ratio of the proton-sequestering agent to the drug is at least 2:1.

3. The method of claim 1, wherein the proton-sequestering agent has a pK that is less than the pH of the aqueous solution.

4. The method of claim 3, wherein absolute difference between the pH and pK is between 0.2 and 5 units.

5. The method of claim 4, wherein the absolute difference between the pH and pK is between 0.5 and 1 unit.

6. The method of claim 4, wherein the absolute difference between the pH and pK is between 1 and 4 units.

7. The method of claim 4, wherein the absolute difference between the pH and pK is between 3 and 5 units.

8. The method of claim 1, wherein the proton-sequestering agent has a pK that is greater than each pKₐ of any bound acidic functional group on the drug.

9. The method of claim 8, wherein the difference between the pK and pKₐ is greater than 0.2 units.

10. The method of claim 1, wherein the molecular weight of the drug is at least five times the molecular weight of the proton-sequestering agent.

11. The method of claim 1, wherein the proton-sequestering agent is selected from the group consisting of amino acids, oligopeptides, short-chain fatty acids, carboxylic acid salts, derivatives thereof, and combinations thereof.

12. The method of claim 11, wherein the proton-sequestering agent is an amino acid or a derivative thereof.

13. The method of claim 12, wherein the amino acid or derivative thereof is selected from the group consisting of glycine, alanine, valine, asparagine, leucine, norleucine, isoleucine, phenylalanine, tryptophan, tyrosine, proline, methionine, acylated forms thereof, and combinations thereof.

14. The method of claim 12, wherein the amino acid is a naturally occurring amino acid.

15. The method of claim 12, wherein the amino acid is leucine.

16. The method of claim 12, wherein the derivative is an acylated derivative of the amino acid.

17. The method of claim 12, wherein the amino acid or derivative thereof is selected from the group consisting of leucine, isoleucine, glycine, alanine, valine, phenylalanine, proline, methionine, glycine, and combinations thereof.

18. The method of claim 11, wherein the proton-sequestering agent is an oligopeptide.

19. The method of claim 18, wherein the oligopeptide is selected from the group consisting of poly-lysine, poly-glutamic acid, and poly-lysine/alanine, dileucine, leu-leu-gly, leu-leu-ala, leu-leu-val, leu-leu-leu, leu-leu-ile, leu-leu-met, leu-leu-pro, leu-leu-phe, leu-leu-trp, leu-leu-ser, leu-leu-thr, leu-leu-cys, leu-leu-tyr, leu-leu-asp, leu-leu-glu, leu-leu-lys, leu-leu-arg, leu-leu-his, leu-leu-nor, leu-gly-leu, leu-ala-leu, leu-val-leu, leu-ile-leu, leu-met-leu, leu-pro-leu, leu-phe-leu, leu-trp-leu, leu-ser-leu, leu-thr-leu, leu-cys-leu, leu-try-leu, leu-asp-leu, leu-glu-leu, leu-lys-leu, leu-arg-leu, leu-his-leu, leu-nor-leu, lsy-lys-lys, and combinations thereof.

20. The method of claim 11, wherein the proton-sequestering agent is a short-chain fatty acid.

21. The method of claim 20, wherein the short-chain fatty acid is selected from the group consisting of tetradecanoic acid, pentadecanoic acid, hexadecanoic acid, octadecanoic acid, eicosanoic acid, docsosanoic acid, tetracosanoic acid, 9-tetradecenoic acid, 9-hexadecenoic acid, 11-octadecenoic acid, 9-octadenoic acid, 1-eicosenoic acid, 5,8,11-eicosatrienoic acid, 13-docosenoic acid, 15-tetracosanoic acid, 9,12,15-octadecatrienoic acid, 6,9,12,15-octadecatetraenoic acid, 11,14,17-eicosatrienoic acid, 8,11,14,17-eicsoatetraenoic acid, 5,8,11,14,17-eicosapentaenoic acid, 7,10,13,16,19-docosapentaenoic acid, 4,7,10,13,16,19-docosahexaenoic acid, 6,9,12,15,18,21-tetracosahexaenoic acid, 9,12-octadecadienoic acid, 6,9,12-octadecatrienoic acid, 11,14-eicosadienoic acid, 8,11,14-eicosatrienoic acid, 5,8,11,14-eicosatetraenoic acid, 13,16-docosadienoic acid, 7,10,13,16-docosatetraenoic acid, 4,7,10,13,16-docosapentaenoic acid, 9-trans-hexadecenoic acid, 9-trans-octadecenoic acid, 8-eicosaenoic acid, 5-eicosaenoic acid, and combinations thereof.

22. The method of claim 11, wherein the proton-sequestering agent is a carboxylic acid salt.

23. The method of claim 22, wherein the carboxylic acid salt comprises salts of carboxylic acids selected from the group consisting of acetate, citrate, formate, fumerate, malate, methanoate, proponate, oxalate, benzoate, and succinate.

24. The method of claim 1, wherein the drug is a therapeutic protein.

25. The method of claim 24, wherein the therapeutic protein is selected from the group consisting of erythropoietin, Factor VIII, Factor IX, prothrombin, thrombin, alpha-1 antitrypsin, alglucerase, imiglucerase, cyclosporin, granulocyte colony stimulating factor, thrombopoietin, alpha-1 proteinase inhibitor, calcitonin, elcatonin, granulocyte macrophage colony stimulating factor, growth hormone, human growth hormone, growth hormone releasing hormone, heparin, low molecular weight heparin, interferon alpha, interferon beta, interferon gamma, interleukin-1 receptor, interleukin-2, interleukin-1, interleukin-1 receptor antagonist, interleukin-3, interleukin-4, interleukin-6, interleukin-7, interleukin-8, interleukin-9, interleukin-10, interleukin-11, interleukin-12, luteinizing hormone releasing hormone, leuprolide, goserelin, nafarelin, buserelin, insulin, pro-insulin, insulin analogues, amylin, C-peptide, somatostatin, octreotride, vasopressin, follicle stimulating hormone, insulin-like growth factor, insulinotrophin, macrophage colony stimulating factor, nerve growth factor, platelet derived growth factor, basic fibroblast growth factor, acidic fibroblast growth factor, stem cell factor, oncostatin M, heparin derived growth factor, herceptin, epidermal growth factor, endothelial cell growth factor, vascular growth factor, thyroxin, tissue growth factor, keratinocyte growth factor, glial growth factor, tumor necrosis factor, endothelial growth factors, parathyroid hormone, glucagon, thymosin alpha 1, IIb/IIIa inhibitor, phosphodiesterase inhibitors, VLA-4 inhibitors, bisphosphonates, respiratory syncytial virus antibody, cystic fibrosis transmembrane regulator gene, deoxyribonuclease, bactericidal/permeability increasing protein, therapeutic monoclonal antibodies, therapeutic polyclonal antibodies, pharmacologically acceptable salts thereof, and combinations thereof.

26. The method of claim 24, wherein the therapeutic protein is selected from the group consisting of such as parathyroid hormone, calcitonin, insulin, interferon, follicle stimulating hormone, luteinizing hormone releasing hormone, leuprolide, growth hormone, pharmacologically acceptable salts thereof, and combinations thereof.

27. The method of claim 1, wherein the total amount of the proton-sequestering agent is less than 30% of the dry weight of the spray-dried, drug-containing particle.

28. The method of claim 1, further comprising the step of mixing an excipient with the aqueous solution, the feed solution, or both.

29. The method of claim 28, wherein the excipient is selected from the group consisting of carbohydrate excipients, inorganic salts, antimicrobial agents, antioxidants, surfactants, buffers, acids, bases, and combinations thereof.

30. The method of claim 29, wherein the excipient is a carbohydrate excipient.

31. The method of claim 30, wherein the carbohydrate excipient is selected from the group consisting of fructose, maltose, galactose, glucose, mannose, sorbose, lactose, sucrose, trehalose, cellobiose, raffinose, melezitose, maltodextrans, dextrans, starches, mannitol, xylitol, lactitol, glucitol, pyranosyl sorbitol, myoinositol, and combinations thereof.

32. The method of claim 1, wherein the proton-sequestering agent comprises less than 30% of the weight of the spray-dried, drug-containing particles.

33. The method of claim 1, wherein the proton-sequestering agent in the spray-dried, drug-containing particles is substantially amorphous.

34. The method of claim 1, wherein the proton-sequestering agent forms an outer layer on at least a portion of the surface of the spray-dried, drug-containing particles.

35. The method of claim 33, wherein substantially all of the outer layer amorphous.

36. The method of claim 32, wherein the substantially all of the outer layer is crystalline.

37. The method of claim 1, wherein the spray-dried, drug-containing particles are suitable for inhalation.

38. A spray-dried particle comprising a drug and a proton-sequestering agent, wherein the particle is comprised of a) a core having an outer surface, wherein the core comprises the drug and a first portion of the proton-sequestering agent, and b) an outer layer covering at least a part of the outer surface, wherein the outer layer comprises a second portion of the proton-sequestering agent and is substantially free of the drug.

39. The particle of claim 38, wherein the drug is present in the particle in an amount of at least about 1% by weight and the total amount of the proton-sequestering agent present in the particle is not greater than about 30% by weight.

40. The particle of claim 38, wherein the drug is present in the particle in an amount of at least about 50% by weight, and the total amount of the proton-sequestering agent present in the particle is not greater than about 30% by weight.

41. The particle of claim 38, wherein substantially all of the drug is in amorphous form.

42. The particle of claim 38, wherein substantially all of the first portion of the proton-sequestering agent in the core is in amorphous form.

43. The particle of claim 38, wherein substantially all of the second portion of the proton-sequestering agent in the outer layer is in amorphous form.

44. The particle of claim 38, wherein substantially all of the second portion of the proton-sequestering agent in the outer layer is in crystalline form.

45. The particle of claim 38, further comprising a transition zone disposed between the core and said outer layer.

46. The particle of claim 45, wherein the transition zone comprises amorphous forms of the proton-sequestering agent, crystalline forms of the proton-sequestering agent, and amorphous forms of the drug.

47. A pharmaceutical formulation comprising a plurality of particles comprised a drug and a proton-sequestering agent, wherein each particle is comprised of a) a core having an outer surface, wherein the core comprises the drug and a first portion of the proton-sequestering agent, and b) an outer layer covering at least a part of the outer surface, wherein the outer layer comprises a second portion of the proton-sequestering agent and is substantially free of the drug.

48. The formulation of claim 47, suitable for inhalation therapy.

49. The formulation of claim 47, further comprising an excipient.

50. The formulation of claim 47, wherein the optional excipient is selected from the group consisting of carbohydrate excipients, inorganic salts, antimicrobial agents, antioxidants, surfactants, and combinations thereof.

51. The formulation of claim 47, wherein the moisture content of the formulation is less than 6% by weight.

52. The formulation of claim 47, wherein the MMAD of the spray-dried, drug-containing particles is in the range between 0.1 μm to 5 μm.

53. The formulation of claim 47, wherein the therapeutic protein is selected from the group consisting of erythropoietin, Factor VIII, Factor IX, prothrombin, thrombin, alpha-1 antitrypsin, alglucerase, imiglucerase, cyclosporin, granulocyte colony stimulating factor, thrombopoietin, alpha-1 proteinase inhibitor, calcitonin, elcatonin, granulocyte macrophage colony stimulating factor, growth hormone, human growth hormone, growth hormone releasing hormone, heparin, low molecular weight heparin, interferon alpha, interferon beta, interferon gamma, interleukin-1 receptor, interleukin-2, interleukin-1, interleukin-1 receptor antagonist, interleukin-3, interleukin-4, interleukin-6, interleukin-7, interleukin-8, interleukin-9, interleukin-10, interleukin-11, interleukin-12, luteinizing hormone releasing hormone, leuprolide, goserelin, nafarelin, buserelin, insulin, pro-insulin, insulin analogues, amylin, C-peptide, somatostatin, octreotride, vasopressin, follicle stimulating hormone, insulin-like growth factor, insulinotrophin, macrophage colony stimulating factor, nerve growth factor, platelet derived growth factor, basic fibroblast growth factor, acidic fibroblast growth factor, stem cell factor, oncostatin M, heparin derived growth factor, herceptin, epidermal growth factor, endothelial cell growth factor, vascular growth factor, thyroxin, tissue growth factor, keratinocyte growth factor, glial growth factor, tumor necrosis factor, endothelial growth factors, parathyroid hormone, glucagon, thymosin alpha 1, IIb/IIIa inhibitor, phosphodiesterase inhibitors, VLA-4 inhibitors, bisphosphonates, respiratory syncytial virus antibody, cystic fibrosis transmembrane regulator gene, deoxyribonuclease, bactericidal/permeability increasing protein, therapeutic monoclonal antibodies, therapeutic polyclonal antibodies, pharmacologically acceptable salts thereof, and combinations thereof.

54. The formulation of claim 47, wherein the therapeutic protein is selected from the group consisting of such as parathyroid hormone, calcitonin, insulin, interferon, follicle stimulating hormone, luteinizing hormone releasing hormone, leuprolide, growth hormone, pharmacologically acceptable salts thereof, and combinations thereof.

55. A method for treating a patient suffering from a condition that is responsive to treatment with a therapeutic drug comprising administering, via inhalation, a therapeutically effective amount of a pharmaceutical formulation of claim 47.
